(19) United States
(12) Patent Application Publication
(10) Pub. No.: US 2011/0189186 A1
(43) Pub. Date: Aug. 4, 2011

(54) AFFECTING BONE RELATED CONDITIONS USING CD68 BLOCKING AGENTS

(75) Inventors: Xu Feng, Birmingham, AL (US); Zhenqi Shi, Birmingham, AL (US); Erin McCoy, Birmingham, AL (US)

(73) Assignee: THE UAB RESEARCH FOUNDATION, Birmingham, AL (US)

(21) Appl. No.: 13/061,471

(22) PCT Filed: Aug. 17, 2009
(86) PCT No.: PCT/US2009/054050

§ 371 Date: Feb. 28, 2011

Related U.S. Application Data
(60) Provisional application No. 61/092,622, filed on Aug. 28, 2008.

(51) Int. Cl. A61K 39/395; G01N 33/569; A61P 35/04; A61P 19/08; A61P 19/10

(52) U.S. Cl. 424/138.1; 435/7.24; 435/7.23

(57) ABSTRACT

Methods of treating, reducing, and/or preventing cancer metastasis to bone using CD68 blocking agents are provided. For example, the methods can be used to reduce breast cancer metastasis to bone. Also provided are methods of treating, reducing, and/or preventing bone resorption in a subject using CD68 blocking agents. For example, the methods can be used to treat osteoporosis.

### CROSS-REFERENCE TO RELATED APPLICATIONS

This application claims the benefit of U.S. Provisional Application Ser. No. 61/092,622, filed Aug. 28, 2008, incorporated by reference in its entirety herein.

### STATEMENT REGARDING FEDERALLY FUNDED RESEARCH

This invention was made with government support under Grant R01 AR47830 awarded by the National Institutes of Health. The United States government has certain rights in the invention.

### BACKGROUND

Cell-bone interaction plays a pivotal role in regulating physiologic processes including bone remodeling, which involves osteoclasts (i.e., the bone-resorbing cells) and osteoblasts (i.e., the bone-forming cells). Cell-bone interaction is also implicated in the pathogenesis of various bone related conditions including, for example, cancer bone metastasis, osteoporosis, and rheumatoid arthritis.

### SUMMARY

Provided are methods and compositions related to treating, reducing, and/or preventing bone related conditions such as cancer metastasis to bone using a CD68 blocking agent.

The provided methods include methods of reducing or preventing cancer metastasis to bone in a subject with cancer, comprising administering an effective amount of a CD68 blocking agent to the subject with cancer.

The methods also include methods of reducing or preventing excessive bone resorption in a subject, comprising selecting a subject in need of reduced bone resorption and administering an effective amount of a CD68 blocking agent to the subject.

Methods of identifying a CD68 blocking agent are also provided. For example, a method of identifying a CD68 blocking agent can comprise contacting a CD68 expressing cell with a test compound in the presence of bone and determining whether attachment of the CD68 cell to the bone is reduced. A reduction in attachment of the CD68 cell to the bone indicates that the test compound is a CD68 blocking agent. Another example method of identifying a CD68 blocking agent comprises contacting a CD68 expressing cell with a test compound in the presence of bone and determining whether bone resorption is decreased. A decrease in bone resorption indicates that the test compound is a CD68 blocking agent.

Also provided are methods of increasing or maintaining bone density in a subject or slowing bone density reduction in a subject are also provided. The methods include selecting a subject in need of increased or maintained bone density, or a subject in need of slowed bone density reduction and administering an effective amount of a CD68 blocking agent to the subject.

Further provided are pharmaceutical compositions comprising a CD68 blocking agent and a pharmaceutical carrier.

### BRIEF DESCRIPTION OF THE DRAWINGS

FIGS. 1A-C are schematic illustrations of a phage display-based approach for isolating adhesion molecules mediating the attachment of osteoclast precursors, primary bone marrow macrophages, (BMMs) onto bone. FIG. 1(A) illustrates an overview of procedures for constructing a phage display library expressing BMM genes and isolating the phage clones with high affinity for bone slices from the library. FIG. 1(B) illustrates the culturing of BMMs on bone slices. FIG. 1(C) illustrates a procedure and conditions for biopanning.

FIGS. 2A-E are photographs of Western blots illustrating the regulation of macrosialin expression in osteoclast precursors. FIG. 2(A) illustrates up-regulation of macrosialin expression in BMMs by mononuclear phagocyte colony-stimulating factor (M-CSF). Nonadherent BMMs were untreated with M-CSF (lane 1) or treated with 40 ng/ml M-CSF for 0.5 h, 2 h, 8 h and 24 h in tissue culture dishes (lanes 2-5). Cells were harvested and lysed for Western analysis with anti-macrosialin/CD68 antibody. The portion of the blot corresponding to β-actin location was probed with anti-β-actin antibody for loading control. The intensity of macrosialin bands were normalized by that of β-actin. The fold change was calculated and compared. The value for BMMs without M-CSF treatment (lane 1) was arbitrarily set as 1. FIG. 2(B) illustrates expression of macrosialin in RAW264.7 (RAW) cells. RAW cells were cultured in tissue culture dish without M-CSF for 24 h (lane 3) or with M-CSF for 0.5 h, 2 h, 8 h, and 24 h (lanes 3-7). Cells were lysed for Western analysis as described in (A). Cell lysate from fresh BMMs (lane 1) and BMMs treated with M-CSF for 24 h (lane 2) were loaded in the Western blot for comparison. FIGS. 2(C-E) illustrate regulation of macrosialin during osteoclast differentiation. In FIG. 2(C), nonadherent BMMs were cultured under osteoclastogenic conditions (40 ng/ml M-CSF and 100 ng/ml RANKL (Receptor Activator for Nuclear Factor κ B Ligand)) for 1, 2, 3, 4, or 5 days (d). Cells were then lysed for Western analysis to examine macrosialin expression as described in FIG. 2(A). In FIG. 2(D), nonadherents BMMs were cultured in presence of 40 ng/ml M-CSF alone for 1, 2, 3, 4, or 5 d. Cells were then lysed for Western analysis as described in FIG. 2(A). In FIG. 2(E), RAW cells were cultured in the presence of 100 ng/ml RANKL for 1, 2, 3, 4, or 5 d. Cells were then lysed for Western analysis to assess macrosialin expression as described in FIG. 2(A).

FIGS. 3(A, B and D) are photographs of cell cultures stained for tartrate resistant acid phosphatase (TRAP) illustrating that macrosialin plays a role in osteoclastogenesis. FIG. 3C is a photograph of a Western analysis illustrating knocking down of macrosialin expression by RNAi in BMMs. FIG. 3(A) illustrates that anti-macrosialin/CD68 inhibits osteoclast differentiation from BMMs. Nonadherent BMMs were cultured under the osteoclastogenic condition (40 ng/ml M-CSF and 100 ng/ml RANKL) in the absence or presence of different concentration of anti-macrosialin/CD68 antibody or control IgG for 5 days (d). The cultures were stained for TRAP (tartrate resistant acid phosphatase). FIG. 3(B) illustrates that anti-macrosialin/CD68 antibody inhibits osteoclast differentiation from RAW cells. RAW cells were cultured under the osteoclastogenic condition (100 ng/ml RANKL) in the absence or presence of different concentrations of anti-macrosialin/CD68 antibody or control IgG for 5 d. The cultures were stained for TRAP. FIG. 3(C) illustrates knocking down of macrosialin expression by RNAi in BMMs. Oligonucleotides containing functional siRNA sequence or scrambled siRNA sequence were cloned into the retroviral vector containing the 5′ siRNA expression modules. Virus was prepared by transfecting the vectors into 293GPG packaging cells and used to infect BMMs. Infected cells were selected by puromycin (2 μg/ml) and lysed for Western analysis or plated for osteoclastogenesis as shown in FIG. 3(D). FIG. 3(D) illustrates that RNAi-mediated suppression of macrosialin impairs osteoclastogenesis. BMMs in which macrosialin expression is suppressed were treated with 40 ng/ml M-CSF and 100 ng/ml RANKL for 5 d. The cultures were then stained for TRAP. The left panel shows osteoclast formation cultures in 3 wells of experimental and control assays while a high power view of a representative area in each well is shown in the right panel.

FIGS. 4(A-D) show photographs of tissue culture and graphs of data quantifying cell numbers from the tissue culture illustrating that anti-macrosialin/CD68 antibody (MSN) inhibits attachment of osteoclast precursors onto an untreated plastic dish and treated tissue culture dish. FIG. 4(A) illustrates results of an attachment assay with BMMs on tissue culture plates. BMMs were cultured in an untreated plastic dish in the presence of M-CSF (40 ng/ml) for 4 d to ensure that macrosialin is highly expressed. Cells were then lifted by 0.02% EDTA in PBS for attachment assay on treated tissue culture dish with different concentration of anti-macrosialin/CD68 antibody (MSN) or control IgG. The top panels show cells attached on the plates. The bottom panel illustrates quantification of the data. To quantify the data, the number of cells in five randomly chosen view areas of the same size were counted and statistically analyzed. FIG. 4(B) illustrates results of an attachment assay with BMMs on untreated plastic plates. BMMs were cultured in untreated plastic dish in the presence of M-CSF (40 ng/ml) for 3 d. Cells were then lifted by 0.02% EDTA in PBS for an attachment assay on treated tissue culture dish with different concentrations of anti-macrosialin/CD68 antibody (MSN) or control IgG. The top panels show cells attached on the plates. The bottom panel illustrates quantification of the data, which was carried out as described in FIG. 4(A). FIG. 4(C) illustrates an attachment assay with RAW cells on tissue culture plates. RAW cells were lifted by scraping and used for an attachment assay on treated tissue culture dish with different concentrations of macrosialin/CD68 antibody (MSN) or control IgG. The top panels show cells attached on the plates. The bottom panel shows quantification of the data, which was carried out as described in FIG. 4(A). FIG. 4(D) illustrates an attachment assay with RAW cells on non-treated plastic plates. RAW cells were lifted by scraping and used for attachment assay on treated tissue culture dish with different concentration of anti-macrosialin/CD68 antibody (MSN) or control IgG. The top panels show cells attached on the plates. The bottom panel illustrates quantification of the data which was carried out as described in FIG. 4(A).

FIG. 5A shows a schematic diagram of a retroviral vector used to infect BMMs to determine whether Macrosialin/CD68 antibody can specifically block attachment of BMMs onto bone slices and shows a graph of data from culturing BMMs in an untreated plastic dish in the presence of M-CSF (40 ng/ml) for 4 d. Cells were then lifted by 0.02% EDTA in PBS for a bone attachment assay in the presence of macrosialin/CD68 antibody (MSN) or control IgG. 2.5×10⁵ cells were mixed with 5 μg anti-macrosialin/CD68 antibody or control IgG in total volume of 50 ul culture medium to perform bone attachment assay. FIG. 5(B) are photographs showing results of bone resorption assays performed in the presence of different concentration (0, 1 and 3 μg/ml) of anti-macrosialin/CD68 antibody (MSN) or control IgG.

FIG. 6(A) is a photograph of a Western analysis illustrating expression of CD68 in breast cancer cells. Cell lysate prepared from MCF7, MDA-MD-231 (231), MDA-MD-435 (435), and MDA-MD-468 (468) was subject to Western analysis with a macrosialin/CD68 antibody. Blots were stripped and re-probed with a β-actin antibody. FIG. 6(B) shows flow cytometry plots illustrating cell surface expression of CD68 in breast cancer cells. Flow cytometric analyses of MCF7, MDA-MD-231, MDA-MD-435, and MDA-MD-468 were performed with control IgG conjugated with phycoerythrin (IgG-PE) and CD68 antibody conjugated with phycoerythrin (α-CD68-PE). FIG. 6(C) is a graph showing data that illustrates attachment of breast cancer cells onto bone. Bone attachment assays were performed with MCF7, MDA-MD-231 (231), MDA-MD-435 (435), and MDA-MD-468 (468). FIG. 6(D) is a graph showing data illustrates blockage of MDA-MD-231 and MDA-MD-435 (435) attachment onto bone by macrosialin/CD68 antibody. Bone attachment assays with MDA-MD-231 (231) and MDA-MD-435 were performed with macrosialin/CD68 antibody or control IgG. * denotes P<0.05.

### DETAILED DESCRIPTION

Cell-bone interaction plays a pivotal role in regulating physiologic processes including bone remodeling, which involves osteoclasts, the bone-resorbing cells, and osteoblasts, the bone-forming cells. Osteoclasts are multinucleated giant cells derived from mononuclear cells of the monocyte/macrophage lineage.

Attachment of osteoclast precursors onto bone is a prerequisite for osteoclast differentiation. In addition, fully differentiated mature osteoclasts also remain attached to resorb bone. Cell-bone interaction is not only involved in the regulation of physiologic processes, but is also implicated in the pathogenesis of various disorders, including tumor bone metastasis. For example, cell-bone interactions are important in cancer bone metastasis, which involves a physical interaction between cancer cells and bone. Abnormal elevation in osteoclast formation/activity is also implicated in the pathogenesis of diseases including osteoporosis, bone erosion in rheumatoid arthritis, and periodontitis.

Macrosialin (the murine homologue of the human CD68), a heavily glycosylated transmembrane protein, is a member of the lamp/lgp family. Macrosialin comprises a large extracellular domain (291-amino acids), a transmembrane domain (TM, 25-amino acids), and short intracellular domain (10-amino acids). Macrosialin/CD68 is highly and preferentially expressed in murine and human macrophages. Macrosialin/CD68 is expressed on the cell surface and also in lysosomes/late endosomes. Macrosialin/CD68 has been postulated to play diverse roles in various cellular processes such as phagocytosis, lysosomal metabolism, and cell-pathogen interaction. Nucleic acid and amino acid sequences for Macrosialin can be accessed via GenBank Accession No. NM 009853. All of the information, including any nucleic acid and amino acid sequences provided for Macrosialin under GenBank Accession No. NM 009853, is hereby incorporated herein in its entirety by this reference.

CD68 is a 110-kD transmembrane glycoprotein that is highly expressed by human monocytes and tissue macrophages. It is a member of the lysosomal/endosomal-associated membrane glycoprotein (LAMP) family. The protein primarily localizes to lysosomes and endosomes with a smaller fraction circulating to the cell surface. It is a type I integral membrane protein with a heavily glycosylated extracellular domain and binds to tissue- and organ-specific lectins or selectins. The protein is also a member of the scavenger receptor family. Scavenger receptors typically function to clear cellular debris, promote phagocytosis, and mediate the recruitment and activation of macrophages. Nucleic acid and amino acid sequences for CD68 can be accessed via GenBank Accession Nos. NM 001040059 and NM 001251 and P34810. All of the information, including any nucleic acid and amino acid sequences provided for CD68 under GenBank Accession Nos. NM 001040059 and NM 001251 and P34810, is hereby incorporated herein in its entirety by this reference.

The cancer cell-bone interaction is an essential event in breast cancer bone metastasis. Macrosialin/CD68 function is a key adhesion molecule mediating the osteoclast precursor-bone interaction as well as the cancer cell-bone interaction. Although CD68 is normally expressed primarily in macrophages and osteoclasts, CD68 is also highly expressed in abnormal cells such as breast cancer cells and CD68 is involved in attachment of breast cancer cells onto bone.

Provided herein are methods related to blocking CD68 function. For example, provided are methods of treating, reducing, and/or preventing cancer metastasis to bone in a subject having cancer comprising administering an effective amount of a CD68 blocking agent to the subject. As used herein, a subject is an individual. A subject includes, but is not limited to, human and non-human animals. The subject may be a mammal (e.g., a human, horse, pig, rabbit, dog, sheep, goat, non-human primate, cow, cat, guinea pig or rodent), a fish, a bird or a reptile or an amphibian. The term does not denote a particular age or sex. A patient refers to a subject with a disease or disorder or suspected of having a disease or disorder. The term patient includes human and veterinary subjects. The subject can have a cancer at risk of metastasis, or can have a cancer that has metastasized, to bone. Optionally, the cancer is breast cancer, prostate cancer, lung cancer, kidney cancer, thyroid cancer, and/or skin cancer.

A CD68 blocking agent is an agent that inhibits or reduces the interaction of CD68 expressing cells with bone, or components of bone. For example, a CD68 blocking agent can be used to inhibit the interaction of an osteoclast expressing CD68, or an osteoclast precursor cell expressing CD68, with bone. CD68 blocking can be assayed with attachment assays and resorption assays, wherein the attachment of CD68 expressing cells to bone is assayed or the resorption of bone is assayed. The term CD68 blocking agent is not meant to suggest that all CD68 functions are blocked. Rather, the function may be reduced sufficiently to be therapeutically useful. Furthermore, other CD68 functions, unrelated to bone resorption or attachment, may not be reduced at all.

A CD68 blocking agent can be used to inhibit the interaction of a cancer cell expressing CD68 and bone. For example, the CD68 blocking agent can inhibit the interaction of a metastatic cancer cell (e.g., a breast cancer cell) with bone. CD68 blocking agents can be used to prevent cancer bone metastasis in a subject, or to reduce metastasis of cancer cells to bone in a subject. Breast, prostate, lung, kidney, thyroid and skin cancer have a high tendency to metastasize to bone, and a CD68 blocking agent can be used to prevent or treat skeletal metastasis in these cancers by mediating the attachment of tumor cells onto bone. Preventing includes delaying the occurrence of metastasis as well as the complete avoidance of metastasis. Treatment includes elimination of the metastasis or a reduction of metastatic tumor size, or a delay in enlargement of the metastatic tumor size.

CD68 blocking agents can also be used in methods of treating, reducing, and/or preventing excessive or pathologic bone resorption in a subject, comprising administering an effective amount of a CD68 blocking agent to the subject. Excessive bone resorption can cause pathologic bone loss wherein the homeostatic balance of bone resorption and formation is disrupted such that bone resorption exceeds bone formation resulting in bone loss. Excessive bone resorption can be caused by a variety of conditions. For example, excessive bone resorption can be caused by osteoporosis conditions, including primary osteoporosis, secondary osteoporosis, neoplastic osteoporosis, gastrointestinal osteoporosis, rheumatologic disease (e.g., rheumatoid arthritis), metastatic cancer, and periodontitis. Excessive bone resorption associated with each of these conditions can be treated, reduced, and/or prevented using an effective amount of a CD68 blocking agent, which is administered to the subject with or at risk of developing excessive bone resorption.

Bone resorption can be measured, evaluated and quantified using known techniques. For example, biochemical markers of bone turnover are products released from osteoblasts and osteoclasts or collagen breakdown products, which can be assessed to measure, evaluate, and/or quantify bone resorption in a subject. Biochemical markers of bone formation include bone-specific alkaline phosphatase (BAP), osteocalcin (OC) and procollagen peptides. All of these can be measured by immunoassay techniques. Bone resorption markers also include tartrate-resistant acid phosphatase (TRAP) and collagen breakdown products, such as pyridinium cross-links, galactosyl hydroxylysine and cross-linked telopeptides, such as CTx and NTx. Thus, optionally, serum BAP and DPD or NTx are assessed to measure, evaluate and quantify bone resorption is a subject. The use of biochemical markers for assessing bone resorption in a subject can be accomplished using methods and markers know to those skilled in the art. For example, markers and techniques for using the markers are described in Swaminathan, R., “Biochemical markers of bone turnover,” Clinica Chimica Acta 313 (2001) 95-105, which is incorporated herein by reference in its entirety. Bone resorption can also be measured, evaluated, and quantified more invasively, for example, by analysis of bone samples taken from the subject.

Bone mineral density (BMD) can also be assessed in a subject. BMD assessment can be used in conjunction with assessment of biochemical markers to correlate bone resorption activity with the subject's bone phenotype. For example, dual-energy X-ray absorptiometry (DXA) instruments can be used to measure bone mineral density (BMD). DXA measures areal BMD (aBMD) in g/cm² by using ionizing radiation with photon beams of two different energy levels. The differences in attenuation of the beams passing through body tissues of variable composition allow the instrument to provide a quantitative measurement of bone density. Quantitative computed tomography (QCT) is another example procedure for measuring, evaluating, and quantifying bone resorption and/or BMD. QCT measures volumetric BMD (vBMD) in mg/cm³ in a standard CT machine. QCT is able to distinguish between cortical and trabecular bone compartments and can measure BMD.

Conventional X-ray and ultrasound techniques can also be used to measure, evaluate, or quantify bone resorption and/or BMD including peripheral dual-energy X-ray absorptiometry (pDXA), peripheral quantitative computed tomography (pQCT), and quantitative ultrasound (QUS).

Thus, the effectiveness of a CD68 blocking agent in the treatment, reducing and/or preventing bone resorption in a subject can be determined using these example techniques. Similar techniques can be used to determine the effectiveness of CD68 blocking agents in the treatment and/or prevention of cancer metastasis to bone. Serum tumor markers and medical imaging can also be used for assessment of cancer metastasis to bone. Tumor markers for assessing bone metastasis and imaging techniques for assessing bone metastasis are know to those skilled in the art.

These and other techniques can also be used to identify a subject in need of treatment with a CD68 blocking agent. Other clinical techniques can also identify a subject in need of treatment, including a subject's medical history, risk factors (e.g. age, other medical conditions, family medical history, medications), and physical examination. Moreover, any method of cancer diagnosis can be used to identify a cancer in a subject that has metastasized to bone or that has a risk for metastasizing to bone. These techniques, alone or in combination, in combination with the imaging and diagnostic techniques described above, or in combination with other techniques for assessing bone characteristics or cancer in a subject can be used to identify a subject in need of treatment with a CD68 blocking agent.

An effective amount of a CD68 blocking agent is a nontoxic but sufficient amount of the blocking agent to provide the desired result (e.g., reduced cancer metastasis, reduced bone resorption, increased bone density, maintained bone density, and/or slowed bone density reduction). The dosages or amounts of the compositions described herein are large enough to produce the desired effect (e.g., reduced cancer metastasis, reduced bone resorption, increased bone density, maintained bone density, and/or slowed bone density reduction) in the method by which delivery occurs. The effective amount can vary from subject to subject, depending on the species, age, and general condition of the subject, the severity of the medical condition or disease that is being treated, the particular agent used, its mode of administration, and the like. Thus, it is not possible to specify an exact effective amount. However, an appropriate effective amount can be determined by one of ordinary skill in the art, for example, by using the assessment techniques indicated above. The dosage should not be so large as to cause adverse side effects, such as unwanted cross-reactions, anaphylactic reactions, and the like. The dosage can be adjusted by an individual physician, veterinarian, or other medical professional based on the clinical condition of the subject involved. The dose, schedule of doses, and route of administration can be varied accordingly.

The efficacy of administration of a particular dose of a CD68 blocking agent according to the methods can be determined by evaluating the particular aspects of the medical history, signs, symptoms, imaging studies, and objective laboratory tests that are known to be useful in evaluating the status of a subject with a disease or at risk of developing a disease. These signs, symptoms, and objective laboratory tests can vary, depending upon the nature and extent of the condition or disease being treated or prevented, as known to one skilled in the art.

For example, if, based on a comparison with an appropriate control group and/or knowledge of the normal progression of the condition or disease in the general population or the particular individual: 1) a subject's physical condition is shown to be improved, 2) the progression of the condition or disease is shown to be stabilized, slowed, or reversed, or 3) the need for other medications for treating the condition or disease is lessened or obviated, then a particular treatment regimen is considered efficacious. For example, reducing or preventing a condition or disease in a subject or in a population indicates efficacy. Such effects could be determined in a single subject (e.g., by reducing loss of bone density or reducing cancerous metastasis to bone) or in a population (e.g., using epidemiological studies).

A CD68 blocking agent can be administered to the subject in a number of ways depending on whether local or systemic treatment is desired, and on the area to be treated. Thus, for example, a CD68 blocking agent can be administered intravenously, subcutaneously, intramuscularly, encapsulated in liposomes or microspheres, as an ophthalmic solution and/or ointment to the surface of the eye, as a nasal spray, as a nebulized solution, or as an aerosol to the nasal cavities or airways. Moreover, a CD68 blocking agent can be administered to a subject vaginally, rectally, intranasally, orally, by inhalation, or by intubation. Optionally, the CD68 blocking agent can be administered by intravenous, subcutaneous, intramuscular, or intraperitoneal injection. A CD68 blocking agent can be prepared in conventional forms, either as liquid solutions or suspensions, solid forms suitable for solution or suspension in liquid, or as emulsions. Optionally, administration can be by slow release or sustained release system such that a constant dosage is maintained.

A CD68 blocking agent can be included in a pharmaceutical composition comprising pharmaceutical carriers, such as a sterile aqueous or non-aqueous solution, suspensions, and emulsions, which can also contain buffers, diluents and other suitable additives. Examples of non-aqueous solvents are propylene glycol, polyethylene glycol, vegetable oils such as olive oil, and organic esters such as ethyl oleate. Aqueous carriers include water, alcoholic/aqueous solutions, emulsions, or suspensions, including saline and buffered media. Solutions include sodium chloride solution, Ringer's dextrose, dextrose and sodium chloride, lactated Ringer's, or fixed oils. Preservatives and other additives can also be present such as, for example, antimicrobials, anti-oxidants, chelating agents, and inert gases and the like.

CD68 blocking agents include a variety of agents (e.g. siRNAs that reduce CD68 expression, small molecules and aptamers that competitively bind CD68 or its receptor, or CD68 antibodies and antibody fragments. One example of a CD68 blocking agent is a macrosialin or CD68 antibody. Such an antibody recognizes both macrosialin and its human homologue CD68. An example macrosialin/CD68 antibody is commercially available from Santa Cruz Biotechnology (Santa Cruz, Calif.). However, such an antibody can be made by one of skill in the art.

A humanized CD68 antibody can be made using recombinant technology. For example, such antibodies can comprise one or more of the complementarity determining regions of the mouse CD68 antibody and one or more framework regions of a human antibody. Thus, a humanized antibody can include one or more or all of the complementarity determining regions of the non-human CD68 antibody and the framework regions of the antibody can be from a human. Other methods can also be used to make a fully human version of a CD68 antibody.

Also provided are methods of identifying a compound (i.e. an agent or a combination of agents) that inhibits or reduces the interaction of a CD68 expressing cell with bone (i.e. a CD68 blocking agent). For example a method of identifying such a compound can comprise contacting a CD68 expressing cell with a test compound in the presence of bone and determining whether attachment of the CD68 cell to the bone is reduced. A reduction in attachment of the CD68 cell to the bone indicates that the test compound inhibits the interaction of the CD68 expressing cell with bone. Similarly, methods of identifying a compound that inhibits resorption of bone are provided that comprise contacting a CD68 expressing cell with a test compound in the presence of bone and determining whether bone resorption is decreased. A decrease in bone resorption indicates that the test compound inhibits resorption of bone.

CD68 blocking agents can selectively bind to CD68. For example, CD68 antibodies can be screened for binding to the CD68 or epitopes thereof. Thus, under designated assay conditions, the specified binding moieties bind preferentially to a particular target antigen (i.e., CD68) and do not bind in a significant amount to other components present in a subject or assay system. Epitopes of CD68 include any determinant capable of specific interaction with a described antibody. Epitopic determinants usually consist of chemically active surface groupings of molecules such as amino acids or sugar side chains and usually have specific three dimensional structural characteristics, as well as specific charge characteristics.

A variety of immunoassay formats may be used to select antibodies that are specifically immunoreactive with a particular amino acid sequence such as CD68 or an epitope thereof. For example, solid-phase ELISA immunoassays are routinely used to select monoclonal antibodies specifically immunoreactive with an antigen. Typically a specific or selective reaction is at a statistically significant level above the background signal. For example, at least 1.5 or twice the background signal or noise or more than 10 to 100 times the background signal or noise is selective. Specific binding between an antibody or other binding agent and an antigen generally means a binding affinity of at least 10⁶ M⁻¹. Further examples of specific binding affinity include, but are not limited to, at least 10⁷ M⁻¹, at least 10⁸ M⁻¹, at least 10⁹ M⁻¹, and at least 10¹⁰ M⁻¹. Specific binding between an antibody or other binding agent and an antigen can also be described in terms of their dissociation constant K_{D}. The antibodies and antibody fragments described can bind with a K_{D} of at least 1 mM, at least 500 μM, at least 300 μM, at least 100 μM, at least 50 μM, at least 30 μM, at least 10 μM, or at least 3 μM.

CD68 blocking antibodies can be monoclonal, polyclonal, or chimeric (including, for example, a humanized antibody). Optionally, the antibody specifically binds the CD68 antigen. CD68 blocking antibodies can also bind bone epitopes that block the interaction of CD68 expressing cells and bone.

Polyclonal antibodies can be prepared by immunizing a suitable animal with a selected antigen. The cells producing antibody molecules directed against the antigen can be isolated from the animal (e.g., from the blood) and, optionally, further purified by well-known techniques, such as panning against an antigen-coated petri dish. Modifications can be utilized as desired to select for surface antibodies rather than secreted antibodies.

Any of the many well known protocols used for fusing lymphocytes and immortalized cell lines can be applied for the purpose of generating a cell producing a surface monoclonal antibody. A nucleic acid encoding a monoclonal antibody heavy and light chain can be identified and isolated by screening an immunoglobulin library (e.g., an antibody phage display library) with the antigen to thereby isolate immunoglobulin library members that bind the antigen. Examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., BioTechnology 9:1370-1372 (1991); Hay et al., Hum Antibod Hybridomas 3:81-85 (1992); Huse et al., Science 246:1275-1281 (1989); Griffiths et al., EMBO J 12:725-734 (1993).

After the desired member of the library is identified, the specific sequence can be cloned into any suitable nucleic acid vector and transfected or otherwise injected into a cell such as a fibroblast. Thus, provided herein is a nucleic acid vector encoding the light and/or heavy chain of an antibody or fragment that blocks CD68. The vector can also encode amino acids operably linked to the antibody sequence as appropriate for the cell which is to express the antibody.

A CD68 blocking agent can be included in a kit. For example, a kit can comprise a container or vial of CD68 blocking agent for administration to a subject. The kit can optionally comprise instructions for preparing the CD68 blocking agent for administration and/or for administering the agent to a subject. The kit can optionally include instruments, such as an injecting device, for administering the CD68 blocking agent.

### Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope except as and to the extent that they are included in the accompanying claims. Efforts have been made to ensure accuracy with respect to numbers (e.g., amounts, temperature, etc.), but some errors and deviations should be accounted for.

### Results

### Macrosialin has a High Affinity for Bone

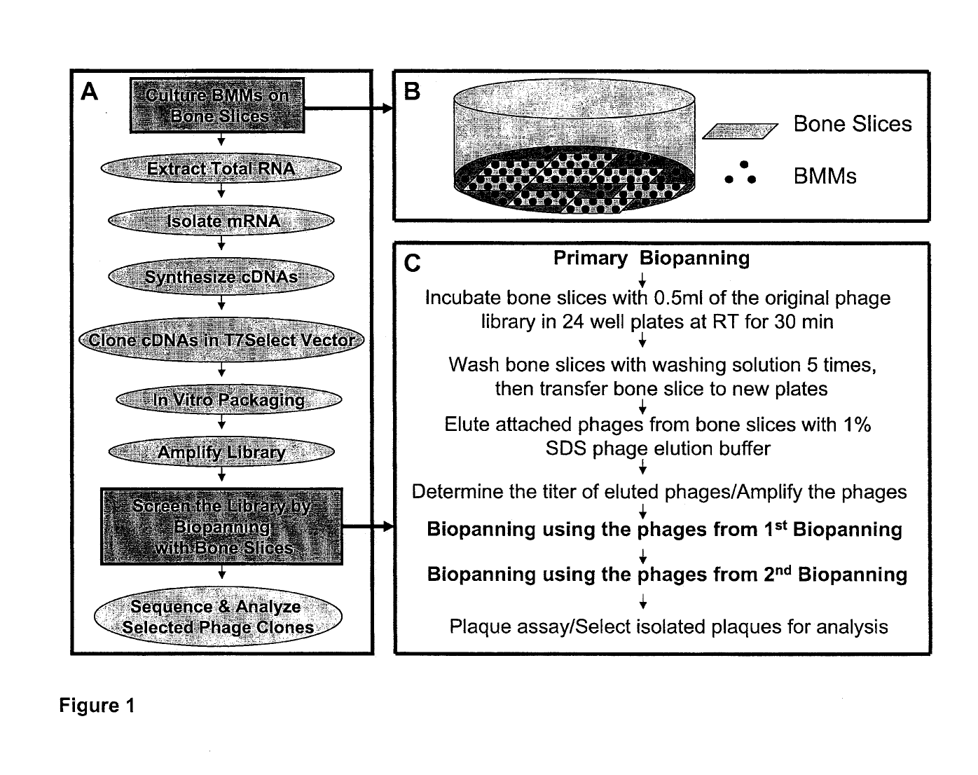

FIG. 1A summarizes steps used to identify phage clones that possess high binding affinity for bone. As shown in FIG. 1B, primary bone marrow macrophages (BMMs), which are, the authentic osteoclast precursors, were cultured on bone slices (prepared from bovine cortical bone) to mimic the normal BMM-bone adhesion condition for 4 days in presence of 40 ng/ml M-CSF. During this period, BMMs not only attached to bone slices but also to the tissue culture dish (FIG. 1B). Bone slices were moved to new tissue culture dishes to ensure that mRNA were isolated only from the cells attached on bone slices to enrich mRNA for the adhesion molecule(s) in the mRNA preparation. The mRNA was used to construct a phage display library following the procedures shown in FIG. 1A. Phages that have high binding affinity for bone were enriched and isolated by three cycles of selection (through biopanning with bone slices) (FIG. 1C).

Thirty-six isolated plaques were randomly selected and PCR was used to determine the insertion of a foreign fragment into gene 10 (which encodes capsid protein displayed on phage shell). Six phages were identified as containing a foreign fragment in gene 10. Among the 6 phages, only one contained an insert encoding mouse protein fragment—macrosialin extracellular domain (a.a. 140-217), while the other 5 phages contained fragments encoding bacterial proteins. Based on the negative control library, approximately 60% of the phages in the library contained either no foreign fragment or fragments resulting from other sources such as contaminated bacterial sources. Not to be bound by theory, the eluted phages containing no inserts or bacterial DNA may have either resulted from non-specific binding or the high affinity of the peptides encoded by the bacterial DNA for bone. Nonetheless, given the features of macrosialin (a transmembrane protein with highly and restricted expression in macrophages) and the availability of numerous commercial antibodies against macrosialin, the involvement of macrosialin in mediating osteoclast-bone interaction was investigated as described below.

### Expression of Macrosialin in Osteoclast Differentiation

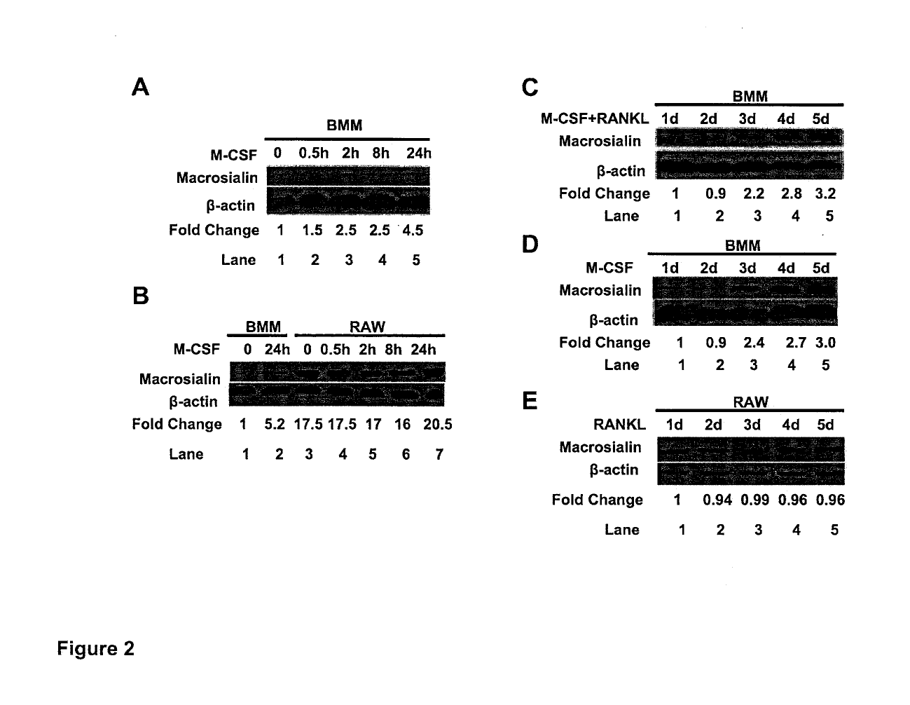

Macrosialin is a heavily glycosylated protein highly and specifically expressed in macrophages. To delineate the role of macrosialin in osteoclast differentiation, macrosialin expression was characterized in osteoclast precursors and during osteoclastogenesis. Freshly isolated BMMs were nonadherent and gradually became attached onto tissue culture dish with stimulation of M-CSF, which plays an important role in survival, proliferation, and differentiation of macrophages. In vitro generation of osteoclasts from BMMs used M-CSF but also RANKL and the entire osteoclastogenic process took 4 to 5 days. As shown in FIG. 2A, macrosialin was barely expressed in BMMs freshly isolated from the bone marrow (lane 1). However, macrosialin expression was up-regulated by M-CSF (40 ng/ml) with time and the 24 h M-CSF treatment increases the macrosialin expression in BMMs about 4.5 fold (FIG. 2A). It is worthwhile to note that BMMs treated with M-CSF for 24 h still remain nonadherent. RAW264.7 (RAW) cells, an immortalized mouse macrophage cell line, were able to quickly attach onto tissue culture plates upon plating. RAW cells expressed high levels of CD68 (lane 3, FIG. 2B), which was much higher than that of BMMs treated with M-CSF for 24 h (lane 2, FIG. 2B). Moreover, as immortalized cells, RAW cell survival and proliferation were independent of M-CSF, indicating that M-CSF-mediated signals were constitutively active. Consistent with this notion, M-CSF treatment did not further increase macrosialin expression in RAW cells (lane 3-7, FIG. 2B).

Next, macrosialin expression was examined during osteoclast differentiation. As control, BMMs were treated with M-CSF (40 ng/ml) alone for 5 days and the macrosialin expression was determined at one-day intervals. Macrosialin expression was increased with time and reached a plateau at day 3 (FIG. 2C), which is comparable to the macrosialin levels seen in RAW cells (FIG. 2B). These data were consistent with observations that BMMs treated with M-CSF started to attach to tissue culture dish one day after M-CSF treatment and most cells became attached to a dish by day 3. In osteoclast differentiation assays, BMMs were treated with M-CSF (40 ng/ml) and RANKL (100 ng/ml). While the macrosialin expression was up-regulated during the 5-day osteoclastogenic process (FIG. 2D), the temporal pattern and magnitude of the macrosialin induction was similar to those seen in the cultures with M-CSF alone (FIG. 2C), indicating that the macrosialin expression during osteoclastogenesis was primarily regulated by M-CSF. This was further supported by the finding that treatment of M-CSF-independent RAW cells had no impact on macrosialin expression (FIG. 2E).

### Macrosialin Plays an Important Role in Osteoclastogenesis

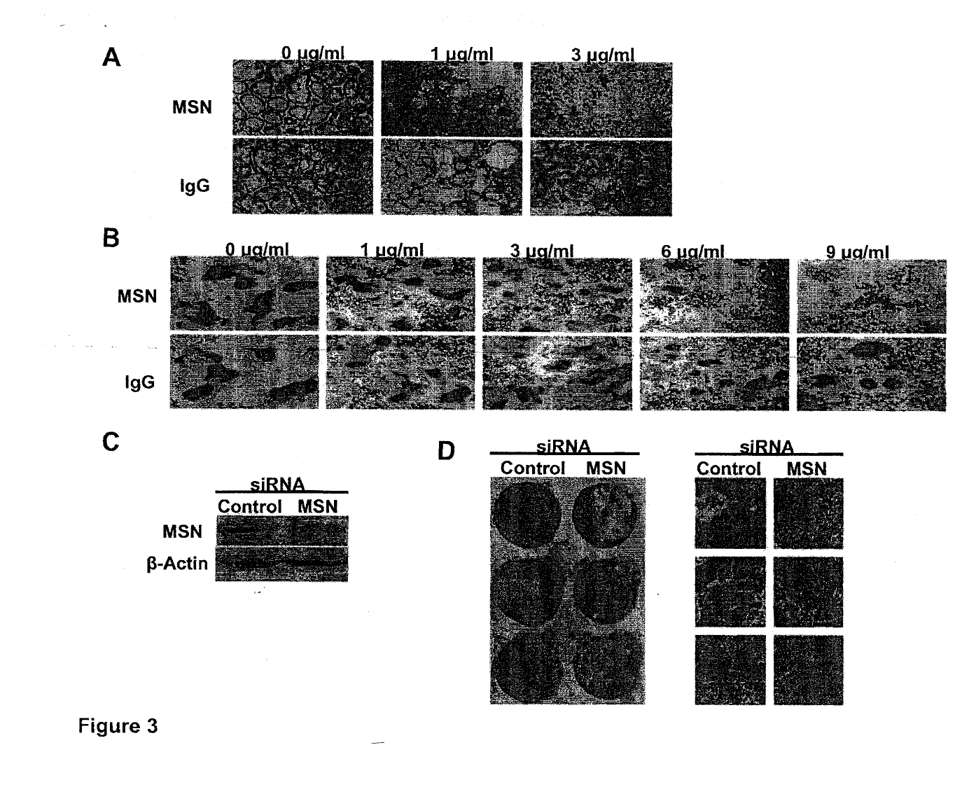

A standard osteoclast formation assay was established by culturing BMMs with M-CSF (40 ng/ml) and RANKL (100 ng/ml). The assays were supplemented with different concentrations of macrosialin/CD68 antibody, which recognizes both macrosialin and its human homologue CD68, or control IgG, both from Santa Cruz Biotechnology (Santa Cruz, Calif.). The data showed that the antibody specifically and potently inhibits osteoclast formation in a dose-dependent manner (FIG. 3A). To further address the issue, RAW cells were treated with 100 ng/ml RANKL to induce osteoclast formation in the presence of different concentration of the anti-macrosialin/CD68 antibody or control IgG. The antibody also inhibited osteoclast formation from RAW cells in a dose-dependent manner (FIG. 3B). Higher concentrations of the antibody were used to block osteoclast formation from RAW cells than from BMMs. This reflects the fact that RAW cells expressed higher levels of macrosialin than BMMs (FIG. 2).

To provide additional evidence supporting an important role for macrosialin in osteoclast formation, the macrosialin expression in BMMs was knocked down by RNAi and the impact of RNAi-mediated CD68 suppression on osteoclastogenesis was investigated. As shown in FIG. 3C, the macrosialin expression was suppressed using the retroviral vector-based siRNA approach described in Wang et al., “Development and validation of vectors containing multiple siRNA expression cassettes for maximizing the efficiency of gene silencing,” BMC Biotechnology 60, 50 (2006). BMMs with suppressed CD68 expression exhibited a dramatically reduced capacity to form osteoclasts in vitro (FIG. 3D). Together, these data indicate that macrosialin plays a critical role in osteoclastogenesis.

Macrosialin Mediates Attachment of Osteoclast Precursors onto Tissue Culture Dish

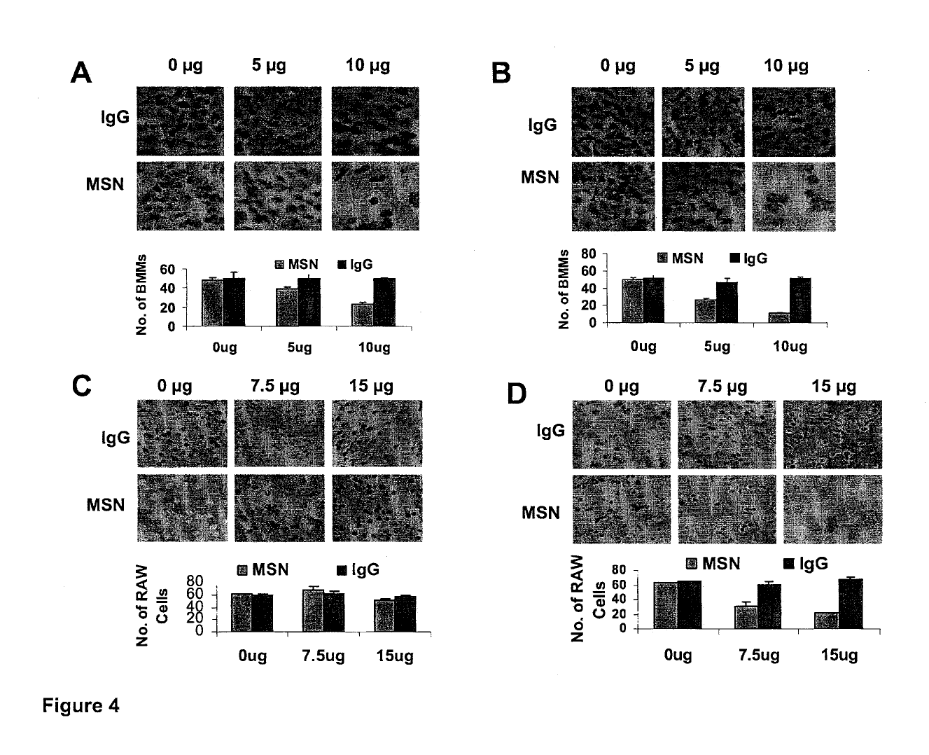

The attachment assays showed that macrosialin/CD68 antibody blocked attachment of BMMs onto tissue culture dish in a dose-dependent manner (FIG. 4A), indicating that macrosialin was involved in mediating attachment of osteoclast precursors onto tissue culture dish. The attachment assays were repeated with untreated plastic dishes. The data indicated that macrosialin/CD68 antibody blocked the attachment of BMMs onto uncoated plastic dish in a dose-dependent manner (FIG. 4B). The antibody more profoundly inhibited the attachment of BMMs onto an untreated plastic dish than tissue culture dish (FIG. 4A-B). Specifically, the highest concentration used (10 μg/ml) gave rise to about 60% inhibition in BMM attachment onto tissue culture plates while the same concentration led to more than 85% inhibition in BMM attachment onto untreated plastic dishes (FIG. 4A-B). These data support that the interaction between BMMs and tissue culture dish was mediated by both macrosialin and integrins while the interaction between BMMs and untreated plastic dish is primarily modulated by macrosialin.

The attachment assays shown in FIG. 4A-B were repeated with RAW cells. Anti-macrosilain/CD68 antibody failed to inhibit the attachment of RAW cells onto tissue culture dish (FIG. 4C), indicating that the interaction between RAW cells and tissue culture dish is predominantly mediated by integrins rather than macrosialin. Nonetheless, the antibody significantly inhibited the interaction between RAW cells and untreated plastic dishes in a dose-dependent manner (FIG. 4D), confirming that macrosialin was involved in the attachment of osteoclast precursors onto untreated plastic dishes. Taken together, these findings indicate that macrosialin was able to interact with inorganic molecular moieties, which are common or similar in bone and plastic.

### Macrosialin Mediates the Osteoclast Precursor-Bone Interaction and is Critical for Osteoclast Function

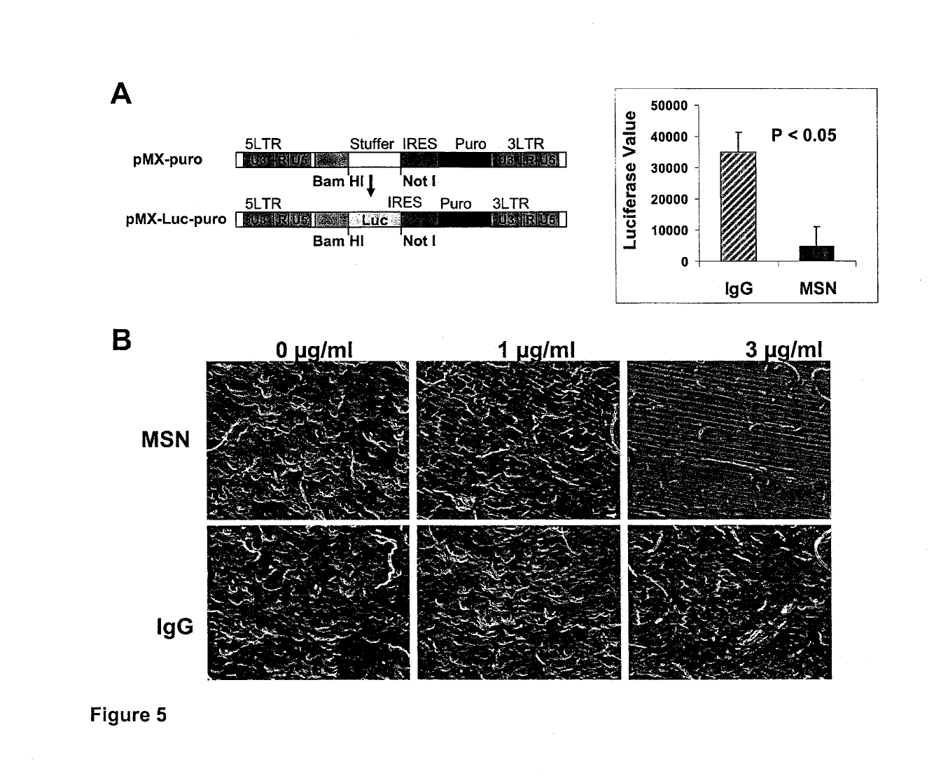

Macrosialin/CD68 antibody or control IgG was used to determine whether the antibody can specifically block the attachment of BMMs onto bone slices. To this end, a retroviral vector named pMX-Luc-puro was prepared by cloning luciferase (Luc) cDNA into pMX-puro vector (between Bam HI and Not I) (FIG. 5A). Then retrovirus was prepared by transiently transfecting 293GPG packaging cells, which are highly efficient packaging cells that can produce high titer viruses (1-2×10⁶ cfu/ml). The virus was used to infect BMMs and positively infected cells were selected with puromycin (2 μg/ml). Cells were mixed with macrosialin/CD68 antibody or control IgG and then added onto bone slices. Cells that were attached onto bone slices were lysed for luciferase activity assay. Luciferase activity was proportional to the number of cells attached. The data indicated that macrosialin/CD68 antibody significantly blocked the attachment (FIG. 5A), supporting that macrosialin and CD68 play a key role in the BMM-bone interaction.

Bone resorption assays in the presence of different concentrations of macrosialin/CD68 antibody or control IgG were performed. The bone resorption assays demonstrated that macrosialin/CD68 antibody specifically inhibits bone resorption in a dose-dependent manner (FIG. 5B), supporting that macrosialin-mediated attachment of BMMs onto bone matrix plays an important role in bone resorption.

Taken together, the data collectively demonstrated that macrosialin is a newly identified molecule that plays a critical role in mediating the attachment of osteoclast precursors on bone and the attachment is important for osteoclast formation and function.

CD68 also Mediates the Interaction Between Bone and Metastatic Breast Cancer Cells

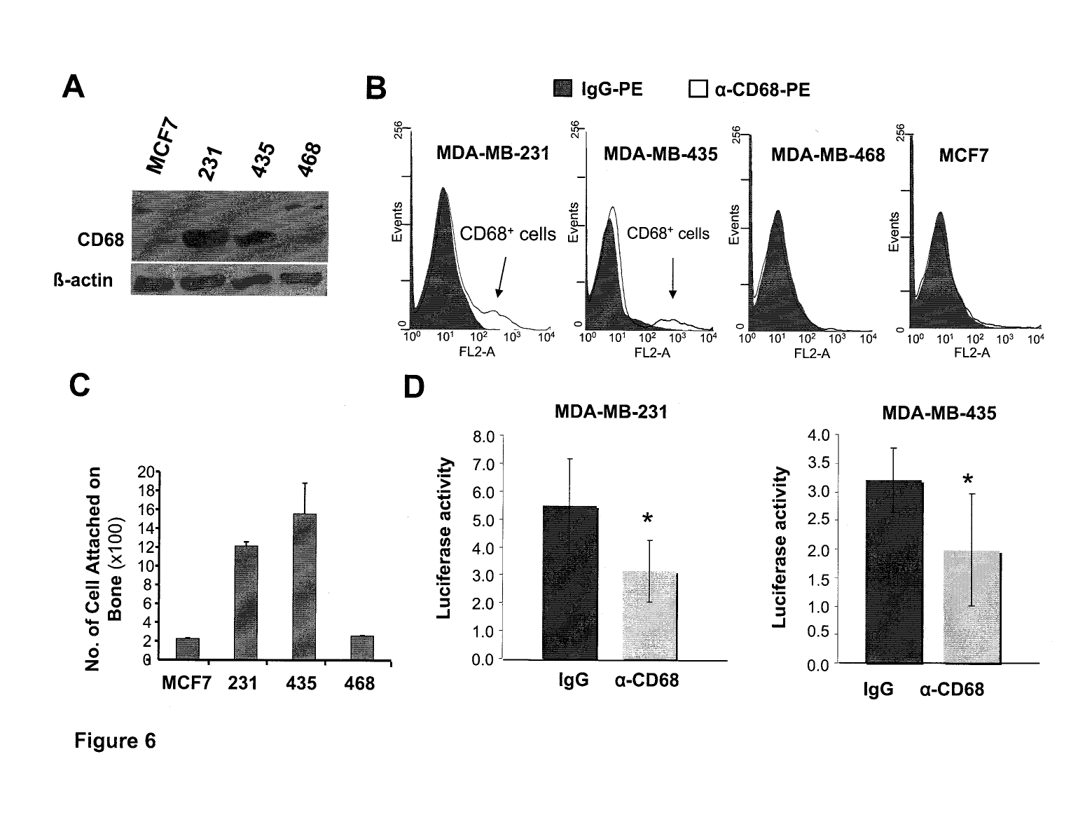

Whether CD68 is expressed in four breast cancer cell lines (MCF7, MDA-MB-231, MDA-MB-435, and MDA-MB-468) was determined. It was found that CD68 is expressed abundantly in MDA-MB-231 and MDA-MB-435, two breast cancer cell lines capable of metastasizing to bone in animal models (FIG. 6A). Flow cytometric analysis demonstrated that subpopulations of MDA-MB-231 and MDA-MB-435 have high levels of CD68 expression on cell surfaces (FIG. 6B). The remaining cells of MDA-MB-435 showed low levels of cell surface expression of CD68 (FIG. 6B). These data further demonstrate a role for CD68 as an adhesion molecule. It was next determined whether the capacity of these breast cancer cells to attach onto bone was positively correlated with their CD68 expression levels. To address this issue, a retrovirus encoding luciferase was prepared by transiently transfecting 293GPG packaging cells with pMX-Luc-puro. The virus was used to infect the four breast cancer cell lines and positively infected cells were selected with puromycin (2 μm/ml). The selected breast cancer cells were used to perform attachment assays. 5×10⁴ cells were incubated with one bone slice for 1 hour and 3 replicates for each cell line were set up. After the incubation, bone slices were washed three times with PBS. Then, bone slices were moved to a new dish and cells attached onto bone slices were lysed for luciferase activity assay. In order to calculate the number of cells attached on bone slices based on luciferase activity counts, a standard curve was prepared for each cell line by plotting different cell numbers (1×10³, 2.5×10³, 5×10³, 7.5×10³, 2.5×10⁴, 5×10⁴) versus the corresponding luciferase activities measured for these cell numbers. The data indicated that MDA-MB-231 and MDA-MB-435 cells, which express abundantly CD68 (FIG. 6A-B), exhibited great capacity to attach to bone (FIG. 6C).

Moreover, it was determined whether macrosialin/CD68 antibody can block the interaction between bone and MDA-MB-231/MDA-MB-435. 5×10⁴ MDA-MB-231 cells were incubated with bone slices for 1 hour with CD68 antibody or control IgG. Bone slices were then washed with PBS and cells attached on bone slices were lysed for luciferase activity assay. The data showed that CD68 antibody blocked attachment of MDA-MB-231 and MDA-MB-435 onto bone (FIG. 6D). Thus, CD68 plays an important role in breast cancer bone metastasis by mediating breast cancer cell attachment on bone.

### Materials and Methods

### Chemicals, Reagents, and Cell Lines

Chemicals were purchased from Sigma (St. Louis, Mo.) unless indicated otherwise. Synthetic oligonucleotides were purchased from Sigma-Genosys (The Woodlands, Tex.). CD68 antibody (Catalog #: sc-9139) and normal rabbit IgG (Catalog #: sc-2027) were from Santa Cruz Biotechnology, Inc (Santa Cruz, Calif.). RAW 264.7 cells, MCF-7, MDA-MB-468, MDA-MB-231, were purchased from ATCC (Manassas, Va.).

### Construction of Phage Display Libraries

Phage display library was constructed using T7Select10-3 OrientExpress cDNA Synthesis from EMD Novagen (Darmstadt, Germany). Total RNA was isolated from BMMs growing on bone slices and mRNA was purified from the total RNA preparation using mRNA Isolation Kit from Miltenyi Biotec (Bergisch Gladbach, Germany). First strand cDNA was synthesized with random primers with two 5′ T's and followed by the second strand synthesis. A linker containing one EcoR I site (5′-GCTTGAATTCAAGC-3′) (SEQ ID NO:1) was added to both ends of cDNA and the addition of the linker to the 3′-end created a Hind III recognition site. cDNA was digested with Eco R I and Hind III and then ligated into directional ends to EcoR/HindIII Vector Arms. The vector was then packaged in vitro using the packaging mixture provided in the kit.

Biopanning with Bone Slices

Bone slices (˜1 cm²) were incubated with 0.5 ml (containing 1.5×10⁶ pfu) of the phage display library in 24 well plates at room temperature for 30 min. Bone slices were washed with washing solution (0.02 M Tris-HCl pH7.6, 0.16 M NaCl, 0.1% TWEEN® (Sigma, St. Louis, Mo.) 5 times and were then transferred to new plates. Attached phages were eluted from bone slices with 200 μl 1% SDS buffer. The eluted phages were tittered and amplified and then used for additional cycles of biopanning

### Osteoclast Formation Assays

BMMs were isolated from long bones of 4-8 week old C3H mice from Harlan Industries (Indianapolis, Ind.) as described in Feng et al., “A Glanzmann's mutation in beta 3 integrin specifically impairs osteoclast function,” J. Clin. Invest 107, 1137-1144 (2001) and cultured in α-MEM containing 10% heat-inactivated FBS in the presence of 40 ng/ml M-CSF. To generate osteoclasts from BMMs, 5×10⁵ cells were plated per well in 24-well plates and cultured in presence of 40 ng/ml M-CSF and 100 ng/ml RANKL or as indicated in individual assays. RAW 264.7 cells (ATCC, Manassas, Va.) were cultured in Dulbecco's Modified Eagle's Medium (DMEM) containing 10% heat-inactivated FBS in tissue culture plates and passed by lifting the cells by scraping. To prepare osteoclasts from RAW 264.7 cells, cells were plated in 24-well plates (3×10⁴/well) and incubated in presence of 100 ng/ml RANKL or as indicated in individual assays. Osteoclasts were stained for TRAP expression with Leukocyte Acid Phosphatase Kit (387-A) from Sigma (St. Louis, Mo.).

### Bone Resorption Assays

Osteoclasts were generated on bovine cortical bone slices in 24-well plates from BMMs with stimulation of M-CSF (40 ng/ml) and RANKL (100 ng/ml) as described. Bone slices were harvested at day 8-9. Cells were removed from bone slices with 0.25 M ammonium hydroxide and mechanical agitation. Bone slices were then subjected to scanning electron microscopy.

### Cell Attachment Assays

BMMs were mixed with anti-CD68 antibody or control IgG in suspension. Cells were then plated on uncoated plastic plates for 15 min and nonadherent cells were removed by washing with PBS 3 times. The cells were fixed with 5% glutaraldehyde (20 min at RT) and washed with water 3 times. 0.1% crystal violet solution was added and plates were incubated for 60 min at RT. After thorough washing with water, cell were visualized and counted.

### Cell-Bone Interaction Assay

A retroviral vector named pMX-Luc-puro was prepared by cloning luciferase (Luc) cDNA into pMX-puro vector. Retrovirus encoding luciferase gene was prepared by transiently transfecting 293GPG packaging cells. The virus was used to infect BMMs or breast cancer cells and positively infected cells were selected with puromycin (2 μg/m). Cells were mixed with anti-CD68 antibody or control IgG and then added onto bone slices. Bone slices with cells were incubated for 15 min (BMMs) or 60 min (breast cancer cells). Then, bone slices were washed with PBS 3 times and the cells attached onto bone slices were lysed for luciferase activity assay.

### Western Blotting

Cells were washed twice with ice-cold phosphate-buffered saline (PBS) and then lysed in buffer containing 20 mM Tris, pH 7.5, 150 mM NaCl, 1 mM EDTA, 1 mM EGTA, 1% Triton X-100, 2.5 mM sodium pyrophosphate, 1 mM β-glycerophosphate, 1 mM Na₃VO₄, 1 mM NaF, and 1× protease inhibitor cocktail 1 (Sigma, P-2850) and 1× protease inhibitor cocktail 2 (Sigma, P-5726). Lysates were then subjected to Western analysis. Membranes were washed extensively and enhanced chemiluminescence (ECL) detection assay was performed using SuperSignal West Dura kit from Pierce (Rockford, Ill.). Quantification of the blots was performed using ImageJ analysis software obtained from NIH (Bethesda, Md.).

### Flow Cytometric Analysis

Parental breast cancer cells lines (MDA-MB-231, MDA-MB-435, MDA-MB-468, and MCF-7) (up to 5×10⁵ cells) were suspended in 200 μl PBS/Azide. Cells were then blocked with 20 μl normal IgG₁ antibody for 30 min on ice. Under dim light, 20 μl of CD68 antibody conjugated with phycoerythrin (Santa Cruz, Calif., sc-17832PE) or control IgG conjugated with phycoerthrin (Santa Cruz, Calif. sc-2866) was added to the cell suspension and cells were incubated on ice for 45 min. Cells were washed twice with 1 ml cold PBS/Azide and resuspended in 300 μl cold PBS/Azide. 200 μl cold 0.5% paraformaldehyde solution was added to fix the cells. Flow cytometric analysis was performed using a Becton-Dickinson FACScan (Becton-Dickinson Immunocytometry Systems, Mountain View, Calif.).

Disclosed are materials, compositions, and components that can be used for, can be used in conjunction with, can be used in preparation for, or are products of the disclosed method and compositions. These and other materials and methods are disclosed herein, and it is understood that when combinations, subsets, interactions, groups, etc. of these materials or steps of a method are disclosed that, while specific reference of each various individual and collective combinations and permutation of these compounds and steps may not be explicitly disclosed, each is specifically contemplated and described herein. For example, if a particular modification of a method of treating a subject having breast cancer with a CD68 blocking agent is disclosed and discussed and a number of modifications that can be made to the method of treating a subject having breast cancer are discussed, each and every combination and permutation of the breast cancer, the CD68 blocking agent, and the treatment method are specifically contemplated unless specifically indicated to the contrary. Likewise, any subset or combination of these is also specifically contemplated and disclosed.

Any patents or publications mentioned in the specification are indicative of the level of those skilled in the art. These patents and publications are herein incorporated by reference in their entirety to the same extent as if each individual publication was specifically and individually indicated to be incorporated by reference.

1. A method of reducing or preventing cancer metastasis to bone in a subject with cancer, comprising administering an effective amount of a CD68 blocking agent to the subject with cancer.

2. The method of claim 1, wherein the cancer is selected from the group consisting of breast cancer, prostate cancer, lung cancer, kidney cancer, thyroid cancer, and skin cancer.

3. The method of claim 1, wherein the cancer is breast cancer.

4. The method of claim 1, wherein the CD68 blocking agent is a CD68 antibody.

5. A method of reducing or preventing excessive bone resorption in a subject, comprising:
(a) selecting a subject in need of reduced bone resorption; and
(b) administering an effective amount of a CD68 blocking agent to the subject.

6. The method of claim 5, wherein the subject has osteoporosis.

7. The method of claim 6, wherein the subject has a condition selected from the group consisting of primary osteoporosis, secondary osteoporosis, neoplastic osteoporosis, gastrointestinal osteoporosis, rheumatologic disease, metastatic cancer, and periodontitis.

8. A method of identifying a CD68 blocking agent, comprising:
(a) contacting a CD68 expressing cell with a test compound in the presence of bone; and
(b) determining whether attachment of the CD68 cell to the bone is reduced, a reduction in attachment of the CD68 cell to the bone indicating that the test compound is a CD68 blocking agent; or
(c) determining whether bone resorption is decreased, a decrease in bone resorption indicating that the test compound is a CD68 blocking agent.

9. The method of claim 8, wherein the CD68 expressing cell is a cancer cell.

10. The method of claim 9, wherein the cancer cell is a breast cancer cell.

11. (canceled)

12. A pharmaceutical composition comprising a CD68 blocking agent and a pharmaceutical carrier.

13. The pharmaceutical composition of claim 12, wherein the CD68 blocking agent is an CD68 antibody.

14. A method of increasing or maintaining bone density in a subject, comprising
(a) selecting a subject in need of increased or maintained bone density; and
(b) administering an effective amount of a CD68 blocking agent to the subject.

15. (canceled)

16. A method of slowing bone density reduction in a subject, comprising
(a) selecting a subject in need of slowed bone density reduction; and
(b) administering an effective amount of a CD68 blocking agent to the subject.


SEQUENCE LISTING (1 sequence)

SEQ ID NO: 1 - DNA, 14 bp, Artificial Sequence
  Synthetic construct
gcttgaattcaagc